(19) United States
(12) Patent Application Publication
(10) Pub. No.: US 2026/0241082 A1
(43) Pub. Date: Aug. 20, 2026

(54) LACTATION DEVICE

(71) Applicants: Diana L. Alatalo, Worcester, MA (US); Phoebe Cutter, Falmouth, MA (US)

(72) Inventors: Diana L. Alatalo, Worcester, MA (US); Phoebe Cutter, Falmouth, MA (US)

(21) Appl. No.: 19/543,093

(22) Filed: Feb. 18, 2026

Related U.S. Application Data
(60) Provisional application No. 63/759,731, filed on Feb. 18, 2025.

(51) Int. Cl. A61M 1/06

(52) U.S. Cl. CPC A61M 1/066; A61M 2205/0216; A61M 2210/1007

(57) ABSTRACT

An interface device insertable into a shield or suction cone of a breast pump provides an alternate engagement angle to the mother for milk extraction that aligns with a natural angle for improved comfort and efficiency in liquid extraction by distributing pressure around the engaging surface. A variable taper in a thickness of the insert material varies the angle of the suction cone to adapt to that of the mother/user. A frustoconical body of the insert shaped to a plateau of reduced or flattened angle around an orifice for liquid extraction, and an inner circumference of the orifice is rounded and/or tapered to align with skin contour. A flexible construction of a deformable material allows sealable engagement between the suction cone and breast surface and an adaptability to form of the mother.

### RELATED APPLICATIONS

This patent application claims the benefit under 35 U.S.C. § 119(e) of U.S. Provisional Patent App. No. 63/759,731, filed Feb. 18, 2025, entitled “LACTATION DEVICE,” incorporated herein by reference in entirety.

### BACKGROUND

Mammals typically nurture newborns with a flow of milk from the mother until sufficient maturity for solid food intake is achieved. In humans, breastfeeding is a natural approach to providing milk for infants through the production of breastmilk that contains essential nutrients necessary for the growth and development of the infant. While natural breastmilk includes a combination of proteins, fats, carbohydrates, vitamins, and minerals beneficial to newborn growth and health, a similar nutritional content may be achieved by manufactured formula which may supplement or substitute for natural breastmilk.

Natural breastmilk may be expressed and stored for a limited time to combine the convenience of portability with the nutritional advantages of natural breastmilk. Commercially available breast pumps are available which purport to express milk from the mother directly into a suitable vessel for storage and subsequent infant administration.

### SUMMARY

An interface device insertable into a shield or suction cone of a breast pump provides an alternate engagement angle to the mother for milk extraction that aligns with a natural angle for improved comfort and efficiency in liquid extraction by distributing pressure around the engaging surface. A variable taper in a thickness of the insert material varies the angle of the suction cone to adapt to that of the mother/user. A frustoconical body of the insert shapes to a plateau of reduced or flattened angle around an orifice for liquid extraction, and an inner circumference of the orifice is rounded and/or tapered to align with skin contour. A flexible construction of a deformable material allows sealable engagement between the suction cone and breast surface and an adaptability to a form of the mother.

Mothers who prefer to avoid infant formula may find logistical challenges in continual breastfeeding, which breast pumps can help mitigate. Configurations herein are based, in part, on the observation that conventional breast pumps employ a single, fixed suction cone that takes the form of a lactation flange or shield designed to encircle and compress against a breast surface for applying reduced pressure for milk extraction. Unfortunately, conventional breast pumps suffer from the shortcomings of a single, fixed angle employed by the shield or flange, typically at an excessively acute angle of approach inconsistent with efficiency and comfort during liquid extraction. Accordingly, configurations herein substantially overcome the shortcomings of conventional lactation flanges by providing an insert for a lactation flange for altering an angle of engagement and improve comfort while the altered angle plateaus to a flattened region around a central orifice for liquid extraction.

Human milk is the recommended food for infants for the first 6 months of life with continued feeding until 2 years of age and beyond. Over 90% of U.S. women initiate breastfeeding after birth. A woman's milk supply develops based on supply and demand; the more milk that is removed results in production of more milk. Thus, frequent and complete emptying of the breast is recommended, particularly during the first month after birth until a woman's milk supply is stable and sufficient for the needs of her infant(s). Additionally, to stimulate the release of prolactin, the hormone responsible for milk production, the nipple of the woman must be rhythmically stretched, which occurs during breastfeeding due to the infant's intraoral vacuum created during suckling. Additional facets of infant suckling that maximize milk removal are angle and shape of mouth. An infant's mouth is oval shaped and attached at a wide angle>120°, which helps conform to the breast shape and avoids occluding milk ducts closer to the surface of the breast. When an infant's mouth is attached at a smaller angle (<120°), less milk is transferred during suckling.

In further detail, configurations herein define a lactation aid device for adapting a breast pump to a user having a frustoconical body configured for insertion in a lactation flange of a breastmilk expressing appliance with an interior surface having a shape configured for defining a circumferential seal with a breast surface. An exterior surface of the frustoconical body is configured for forming a seal with the lactation flange. A central orifice provides for passage of lactated fluids into a collection vessel in response to a low pressure extraction source and simultaneous sealing of the frustoconical body along the interior surface and the exterior surface.

### BRIEF DESCRIPTION OF THE DRAWINGS

The foregoing and other objects, features and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a context diagram of the interface device as disclosed herein;

FIG. 2 is an exploded view of the interface device of FIG. 1 engaged in a usage scenario;

FIGS. 3A-3B are schematic views of the interface device of FIGS. 1 and 2; and

FIGS. 4A-4B show various alternate configurations of the interface device of FIGS. 1-3B.

### DETAILED DESCRIPTION

Depicted below are example configurations of the insertable interface device operable with a flange of a breast pump or suction appliance for expressing natural breast milk for subsequent storage and administration (feeding) to an infant. The interface device, or insert is suitable for sealable insertion into an operation end of a commercially available breast pump device, referred to as a suction cone, lactation flange, breast shield or simply flange or shield.

The interface device or insert mimics a baby's latch and improves user comfort while maintaining effective milk expression. In operation, the disclosed insert mimics and/or supplements the natural breastfeeding process, where a baby's mouth opens to an angle of about 120 to 140 degrees during a proper latch. Shaped inserts are designed to improve ergonomics and comfort for users. A particular configuration employs a 135° angle, which closely simulates natural breastfeeding, and is crafted from soft, skin-benign polydimethylsiloxane (PDMS).

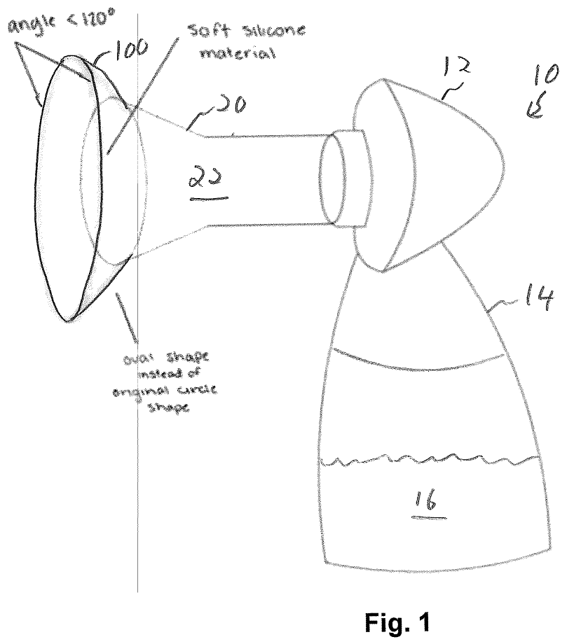

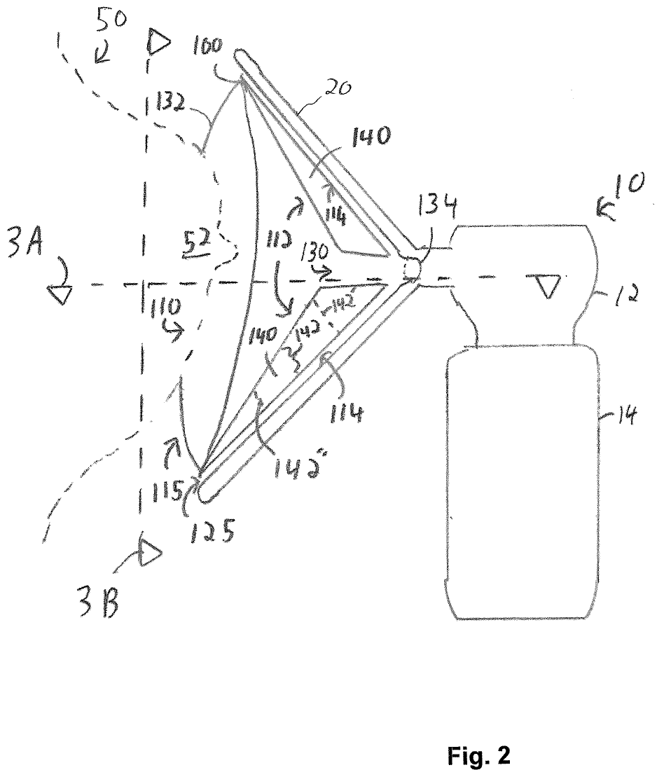

FIG. 1 is a context diagram of the interface device 100 as disclosed herein. Referring to FIG. 1, a breast pump appliance 10 generally includes a pump 12 or motorized head capable of generating low pressure for suction. A vessel 14 connects to an output of the pump 12 and is suitable for storage and/or transport of expressed liquid 16 (breast milk). The pump 12 connects to a lactation flange 20 for forming a region 22 of low pressure for expressing milk from the mother/user. The interface device 100 engages with the lactation flange 20 in a sealable manner in conjunction with the breast surface (as shown below in FIG. 2) for increased efficiency and comfort, generally by adapting a larger angle (greater than 120°) more suited to the human form, although this angle may be adjusted through construction of the interface device 100 and deformability of the device 100.

The insert 100 aims to more closely mimic the natural breastfeeding process, where a baby's latch plays a vital role in milk expression. During breastfeeding, the baby's lips should be flanged (rolled out), and the mouth should open to an angle of around 140°, allowing the baby to latch onto not just the nipple, but also most of the areola. Similarly, prevailing views suggest that a good latch involves the lips at an angle greater than 120°, with the lower lip covering more of the areola. These findings highlight the importance of a wide mouth opening to ensure deep latching and optimal milk flow, without discomfort or misshaping of the nipple.

Traditional breast pumps often have a fixed 90-degree angle, which can limit the user's ability to achieve comfortable and efficient milk expression. Pumps with fixed or non-adjustable angles may result in poor positioning, potentially leading to less effective milk expression and discomfort over time. Conventional approaches have employed angular orientations in the 105° angle range, which has been shown to improve milk flow, as it may better align with the natural latch. However, based on biomimicry, it is expected that babies naturally open their mouths to an angle closer to 120°-140° degrees, which facilitates a more effective milk expression.

The use of breast pumps to express milk and maintain lactation is beneficial for women who must be separated from their infants for extended times or are experiencing difficulty feeding their infants at the breast. In the U.S., approximately 25% of women return to work within two weeks after giving birth, and 90% of women return within one year. The use of breast pumps by working women is common and should result in regular emptying of the breast and rhythmically stretching of the nipple to maintain an adequate milk supply. Additionally, for women who are at high risk of early weaning or delayed secretory activation (mature milk “coming in”), breast pumping within the first hour after birth is recommended with continued use multiple times daily.

In an example configuration, the interface device 100 for a breastfeeding pump includes a generally frustoconical body configured for insertion in a lactation shield of a breast milk extraction device or “breast pump.” The insert features an interior surface in the frustoconical body based on a natural lactation angle of feeding infants, and a central orifice for passage of lactated fluids into a vessel in response to a low-pressure extraction source. A void or opening at a larger diameter end of the frustoconical body appears as a “cup” that engages the user/mother, and a flattened region at a smaller diameter end of the frustoconical body is configured for human engagement for lactation. The smaller diameter end of the frustoconical body is configured for insertion into the flange of a breastfeeding pump.

The frustoconical body generally forms or defines an angle of a cone shape based on emulating an angle of an infant latch of a feeding position; in an example configuration, the angle is between 120°-140°. The frustoconical body may be formed from PDMS (Polydimethylsiloxane) or other suitable material, extruded or molded from a 3-dimensional printed, molding or additive manufacturing printing apparatus.

The frustoconical body 110 may define any suitable angle between the smaller circumference and the larger circumference, such that the angle based on a shape of the breast surface and/or the lactation flange 20 angle. Preferably, the interior surface in the frustoconical body forms an angle based on a natural lactation angle of feeding infants.

FIG. 2 is an exploded view of the interface device of FIG. 1 engaged in a usage scenario. Referring to FIGS. 1 and 2 lactation aid interface device 100 for adapting a breast pump 10 to a user 50 includes a frustoconical body 110 configured for insertion in a lactation flange 20 of a suitable breastmilk expressing appliance such as the breast pump 10. The structure of the frustoconical body 110 includes an interior surface 112 having a shape configured for defining a circumferential seal 115 with a breast surface 52, and an exterior surface 114 configured for forming a seal 125 with the lactation flange 20. The insert device 100 forms a sealable compression between the surface 52 and the lactation flange 20 from the low pressure of the pump 12 and the interior surface 112 contacting the surface 52 concurrently with the exterior surface 114 contacting the lactation flange 20. Thus, frustoconical body 110 defines a continuous deformable material configured for a sealing engagement between the lactation flange 20 and the breast surface 52 for effectively forming a low pressure vessel between the breast pump 10 and the breast surface 52.

The device 100 includes a central 130 orifice for passage of lactated fluids into the vessel 14 in response to a low pressure extraction source from the pump 12 and simultaneous sealing of the frustoconical body 110 along the interior surface 112 and the exterior surface 114.

In technical terms, a frustoconical object is shaped as a cone with the top (apex) removed by a plane parallel to the base. This creates a 3D shape with two parallel circular, or roughly circular, bases of different diameters. It is often described as a tapering or conical shape with a flat, truncated top. In the claimed approach, the frustoconical body 110 has a larger circumference 132 (or diameter) end and a smaller circumference (or diameter) 134 end, where the central orifice 130 is continuous with the smaller circumference 134 end. The larger circumference 132 defines an opening configured for engaging the breast surface 52, and the central orifice 130 defines an opening for fluidic passage to the breast pump 10.

A paramount feature of the device 100 is adaptability between varying shapes and angles of the lactation flange 20 and an optimal or natural angle of the breast surface 52, which may vary. The generally circular appearance of the frustoconical body 110 may be varied to an elliptical form from tapering of material thickness of the device 100. The frustoconical body 110 is generally shaped from a flexible planar material 140 having a thickness, such that the thickness defines a taper between the larger circumference and the smaller circumference for defining a respective elliptical dimension of the smaller circumference 134 and the larger circumference 132. In other words, the larger circumference 132 where the device engages the user may have regions of lesser thickness 142″ over regions of greater thickness 142′, generally at a diametrically opposed, 180° offset for forming an ellipse.

The breast pump flange 20 or shield is a cone shaped component that comes into direct contact with the breast and is used with a pump to create a negative pressure (vacuum) environment that rhythmically varies in imitation of infant suckling. Proper fit of flanges is critical for comfort and effective milk removal. The majority of flanges are circular in shape and have an attachment angle <120°. Since improved milk removal occurs when the flange imitates natural infant attachment, it would be advantageous to provide a device that modifies an existing flange to interface with the breast in the shape and angle of an infant's mouth, namely an oval shape and angle>120°. Varying the angle and material 140 thickness allows for a device customized for women with abnormal breast shapes. Furthermore, it would be advantageous if such a device provided a more comfortable interface while maintaining vacuum pressure and rhythmic nipple stretching using any suitable breast pump.

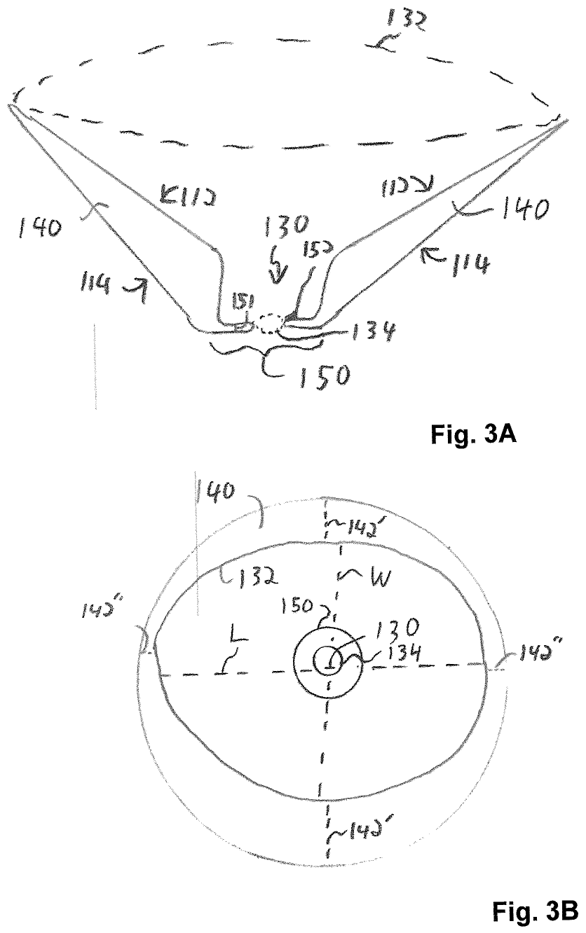

FIGS. 3A-3B are schematic views of the interface device of FIGS. 1 and 2. FIG. 3A shows a cutaway side view, normal to an axial flow through the device 100, and FIG. 3B shows an axial elevation into the larger circumference 132 depicting the thickness variation for forming an elliptical shape. FIG. 3A depicts the central orifice formed in a tapered end 150 of the frustoconical body 110 converging to the central orifice 130. The tapered end 150 may represent a reduced curvature or angle, and may converge to a flat or planar orientation. It should be noted that this convergence generally corresponds to the areola region where the breast surface 52 may vary. An inner circumference 152 of the orifice 130 may be rounded for comfort, and a thickness 151 at the tapered end may be reduced.

Similarly, thickness augmentations progressing towards the larger circumference 132 transition from a circle to an ellipse of the larger circumference having a differing length L and width W, such that the length and width based on a variance of the thickness of the material. A reducing thickness 142″ progressing towards the larger circumference tends to promote a wider angle at the surface 52, while an increasing thickness 142′ emphasizes the width W of an elliptical form. Variance of the thickness 142 at varied regions around the circumference 132, 134 and in progression between the smaller 134 to larger circumference 132 define the respective elliptic and angular properties. It should be noted that as the radial foci of an ellipse coincide, the ellipse becomes circular.

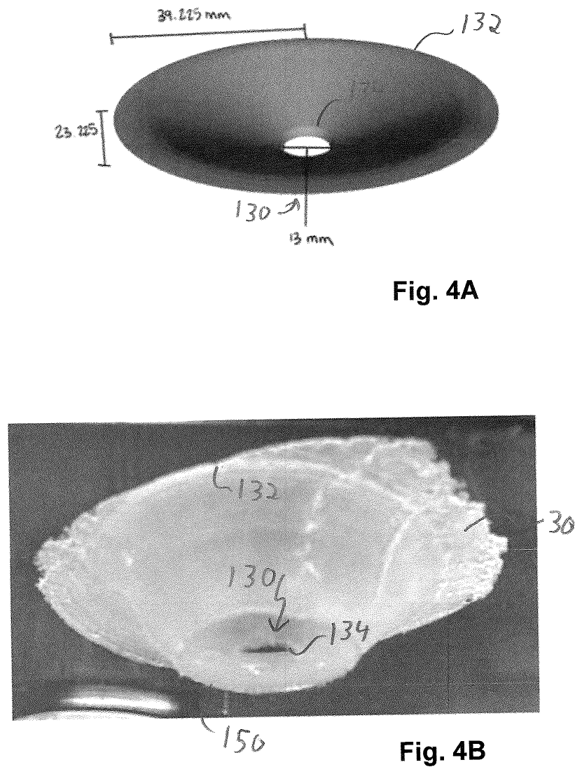

FIGS. 4A-4B show various alternate configurations of the interface device of FIGS. 1-3B. FIG. 4A shows a generated dimensional rendering of a particular configuration with uniform circular transitions from the larger circumference 132 to the smaller circumference 134. FIG. 4B shows a molded version with the tapered end 150 approaching the orifice 130 with excess 30 molded material from runout.

While the system and methods defined herein have been particularly shown and described with references to embodiments thereof, it will be understood by those skilled in the art that various changes in form and details may be made therein without departing from the scope of the invention encompassed by the appended claims.

What is claimed is:

1. A lactation aid device for adapting a breast pump to a user, comprising:
a frustoconical body configured for insertion in a lactation flange of a breastmilk expressing appliance;
an interior surface in the frustoconical body, the interior surface having a shape configured for defining a circumferential seal with a breast surface; and
an exterior surface of the frustoconical body, the exterior surface configured for forming a seal with the lactation flange; and
a central orifice for passage of lactated fluids into a vessel in response to a low pressure extraction source and simultaneous sealing of the frustoconical body along the interior surface and the exterior surface.

2. The device of claim 1 wherein the frustoconical body has a larger circumference end and a smaller circumference end, the central orifice continuous with the smaller circumference end.

3. The device of claim 2 wherein the larger circumference defines an opening configured for engaging the breast surface, and the central orifice defines an opening for fluidic passage to a breast pump.

4. The device of claim 2 wherein the central orifice is formed in a tapered end of the frustoconical body converging to the central orifice.

5. The device of claim 2, wherein the frustoconical body is shaped from a material having a thickness, the thickness defining a taper between the larger circumference and the smaller circumference for defining a respective elliptical dimension of the smaller circumference and the larger circumference.

6. The device of claim 3 wherein the frustoconical body further comprises a continuous deformable material configured for a sealing engagement between the lactation flange and the breast surface for forming a vessel between breast pump and the breast surface.

7. The device of claim 5 wherein the ellipse of the larger circumference has a length and width, the length and width based on a variance of the thickness of the material.

8. The device of claim 2 wherein the frustoconical body defines an angle between the smaller circumference and the larger circumference, the angle based on a shape of the breast surface.

9. The device of claim 2 wherein the interior surface in the frustoconical body forms an angle based on a natural lactation angle of feeding infants.

10. The device of claim 5 wherein the lactation flange has an angle, and a thickness of the material tapers along the frustoconical body for aligning an angle of the interior surface around the smaller circumference with an angle of the exterior surface at the larger circumference, the alignment forming a sealing engagement between the lactation flange and the breast surface.

11. The device of claim 1 further comprising
a void at a larger diameter end of the frustoconical body;
a flattened region at a smaller diameter end of the frustoconical body;
the flattened region configured for human engagement for lactation.

12. The device of claim 1 further comprising:
a void at a larger diameter end of the frustoconical body; and
a smaller diameter end of the frustoconical body, the smaller diameter end configured for insertion into a breastfeeding pump defining the lactation flange.

13. The device of claim 1 wherein the frustoconical body defines an angle of a cone shape based on emulating an angle of an infant latch of a feeding position.

14. The device of claim 8 wherein the angle is between 120°-140°.

15. The device of claim 1 wherein the frustoconical body is formed from PDMS (polydimethylsiloxane).

16. The device of claim 1 wherein the frustoconical body is formed from an extruded filament.
